(19) United States
(12) Patent Application Publication
(10) Pub. No.: US 2012/0284871 A1
(43) Pub. Date: Nov. 8, 2012

(54) PROTEIN EXPRESSION SYSTEMS

(75) Inventors: George Peter Lomonossoff, Norwich (GB); Pooja Saxena, Norwich (GB); Yulia Alexandrovna Meshcheriakova, Norwich (GB)

(73) Assignee: PLANT BIOSCIENCE LIMITED, Norwich (GB)

(21) Appl. No.: 13/464,220

(22) Filed: May 4, 2012

(30) Foreign Application Priority Data
May 4, 2011 (GB) 1107468.9

(51) Int. Cl. C12N 15/63; A01H 5/00; C12N 5/10; C12N 1/19; C12P 21/00; A01H 1/00

(52) U.S. Cl. 800/278; 435/320.1; 435/69.1; 435/419; 435/254.2; 435/348; 435/325; 800/298

(57) ABSTRACT

Disclosed herein are methods and materials, and particularly viral derived sequences, for boosting gene expression in plants and other eukaryotic cells. The methods and materials may be used for boosting expression of heterologous genes encoding proteins of interest.

### FIELD OF THE INVENTION

The present invention relates generally to methods and materials, and particularly viral derived sequences, for boosting gene expression in plants and other eukaryotic cells, for example of heterologous genes encoding proteins of interest.

### BACKGROUND OF THE INVENTION

10

Comoviruses (CPMV)

Comoviruses are RNA viruses with a bipartite genome. The segments of the comoviral RNA genome are referred to as RNA-1 (5889 nucleotides) and RNA-2 (3481 nucleotides). RNA-1 encodes the VPg, replicase and protease proteins (Lomonossoff & Shanks, 1983). The replicase is required by the virus for replication of the viral genome. The RNA-2 of the comovirus cowpea mosaic virus (CPMV) encodes a 58K and a 48K protein, as well as two viral coat proteins L and S.

Initiation of translation of CPMV RNA-1 occurs from a single AUG at position 207 on the RNA and terminates at position 5805, giving a 5′ untranslated region (UTR) of 206 nucleotides and a 3′ UTR of 82 nucleotides. By contrast initiation of translation of the RNA-2 of all comoviruses occurs at two different initiation sites located in the same triplet reading frame (AUGs 161 and 512) and terminates at 3299, resulting in the synthesis of two carboxy coterminal proteins. This double initiation phenomenon occurs as a result of ‘leaky scanning’ by the ribosomes during translation.

Van Bokhoven et al (1993) made heterologous sequence insertions at different positions in the open reading frame of RNA-1 (termed “B-RNA” therein) leaving the 5′ and 3′ UTRs intact. The experiments were performed to investigate the cis- and trans-acting elements required in cowpea mosaic virus RNA replication. Using a T7 polymerase in vitro expression system, the authors reported that none of their mutant RNA-1 sequences were able to replicate when transfected into cowpea protoplasts.

CPMV Vectors

CPMV has served as the basis for the development of vector systems suitable for the production of heterologous polypeptides in plants (Sainsbury et al., 2010).

All the current systems are based on the modification of RNA-2 but differ in whether full-length or deleted versions are used. A key reason why all existing CPMV-based vectors have been based on RNA-2 is because RNA-2 encodes the virus coat proteins (L and S) which are present in 60 copies each per virus particle. By contrast, RNA-1 encodes proteins with catalytic activities (such as the virus-encoded 24K proteinase and polymerase) which need to be present only in much lower amounts. For this reason it is considered that the mRNA encoding the viral coat proteins (RNA-2) must be translated with much greater efficiency, to allow for the discrepancy in the amounts of product required (Fraenkel-Conrat and Kimball, 1982).

For example in one system based on a deleted version of CPMV RNA-2, the region of RNA-2 encoding the movement protein and both coat proteins has been removed. However, the deleted molecules still possess the cis-acting sequences necessary for replication by the RNA-1-encoded replicase and thus high levels of gene amplification are maintained without the concomitant possibility of the modified virus contaminating the environment. With the inclusion of a suppressor of gene silencing in the inoculum in addition to RNA-1, the deleted CP MV vector can be used as a transient expression system (WO/2007/135480). However, in contrast to the situation with a vector based on full-length RNA-2, replication is restricted to inoculated leaves.

However, it has been found that mutation of the start codon at position 161 in a CPMV RNA-2 vector strongly increases the levels of expression of a protein encoded by a gene inserted after the start codon at position 512. This permits the production of high levels of foreign proteins without the need for viral replication and is termed the CPMV-HT system (WO2009/087391; Sainsbury and Lomonossoff, 2008).

The CPMV-HT system was subsequently refined through the creation of the pEAQ series of expression plasmids (Sainsbury et al., 2009). In these plasmids, the sequence to be expressed is positioned between the 5′UTR and the 3′ UTR in single step using either restriction enzyme or Gateway-based cloning.

Thus, known CPMV based vector systems represent useful tools for the expression of a heterologous gene encoding a protein of interest in plants. However, there is still a need in the art for optimised vector systems which can complement or provide modified properties compared to the existing vectors.

### SUMMARY OF INVENTION

Described herein are novel expression systems based on CPMV RNA-1 derived UTRs. The present inventors have surprisingly found that these can give very high and rapid expression levels in transient expression assays.

This RNA-1 based expression system has been referred herein as “CPMV-RT” which stands for Rapid Trans, reflecting the kinetics of expression.

Thus the present invention relates to novel protein production systems and methods, based on modified bipartite virus RNA-1 sequences.

Various aspects of the invention employ RNA-1-derived translational enhancer sequences. A preferred embodiment is the 5′ UTR of CPMV RNA-1. Other preferred RNA-1 enhancer sequences are discussed below.

Thus in one aspect there is provided a gene expression system comprising:

(a) a translational enhancer sequence as described above; and (b) a gene encoding a protein of interest, wherein the gene is located downstream of the enhancer sequence.

The gene expression systems of the invention are nucleic acids, and are typically DNA. It will be readily appreciated by those skilled in the art that where a DNA molecule is said to include an RNA-derived UTR sequence, the DNA sequence will have T in place of U.

The gene and protein of interest operably linked to the enhancer will be heterologous i.e. the expressed sequence will not be exactly that naturally expressed by the wild-type bipartite RNA virus from which the enhancer sequence is derived. To put it another way, the sequence 3′ to the enhancer sequence will not be that naturally occurring in the RNA-1 genome of the wild-type bipartite RNA virus.

More preferably the translated sequence will not encode any of the proteins naturally encoded by the RNA-1 genome of the wild-type bipartite RNA virus.

More preferably the sequence 3′ to the enhancer sequence will not encode (in or out of frame) any of the proteins naturally encoded by the RNA-1 genome of the wild-type bipartite RNA virus.

More preferably the translated sequence will not include any of the proteins naturally encoded by the RNA-1 or RNA-2 genomes of the wild-type bipartite RNA virus. Optionally it may also not encode any CaMV proteins.

The gene expression systems of the invention may thus be used to express a protein of interest in a host organism. In this case, the protein of interest may also be heterologous to the host organism in question i.e. introduced into the cells in question (e.g. of a plant or an ancestor thereof) using genetic engineering, i.e. by human intervention. A heterologous gene in an organism may replace an endogenous equivalent gene, i.e. one which normally performs the same or a similar function, or the inserted sequence may be additional to the endogenous gene or other sequence.

Persons skilled in the art will understand that expression of a gene of interest will require the presence of an initiation site (AUG) located upstream of the gene to be expressed. Such initiation sites may be provided either as part of an enhancer sequence or as part of a gene encoding a protein of interest.

The host cell or organism may be a plant or a plant cell line—for example the well known tobacco BY-2 cell line (see “Tobacco BY-2 Cells”, Edited by Nagata, Toshiyuki; Hasezawa, Seiichiro; Inzé, Dirk Springer 2004).

Plants in this context includes both lower (e.g. bryophytes, such as mosses, and algae) and higher (vascular) plants. However, as translational mechanisms are well conserved over eukaryotes, the gene expression systems may also be used to express a protein of interest in eukaryotic host organisms other than plants, for example in insect cells as modified baculovirus vectors, or in yeast or mammalian cells.

Gene expression systems will typically be operably linked to promoter and terminator sequences. In embodiments of the invention, the promoter may be an inducible promoter.

Thus, gene expression systems may further comprise a termination sequence and the gene encoding a protein of interest may be located between the enhancer sequence and the termination sequence, i.e. downstream (3′) of the enhancer sequence and upstream (5′) of the termination sequence.

The gene expression system may be in the form of an expression construct or expression cassette.

Thus the invention further provides an expression cassette comprising:

(i) a promoter, operably linked to

(ii) an enhancer sequence as described above

(iii) a gene of interest it is desired to express

(iv) a terminator sequence.

Gene expression cassettes, gene expression constructs and gene expression systems of the invention may also comprise a 3′ untranslated region (UTR).

The 3′UTR may be located upstream of a terminator sequence present in the gene expression cassette, gene expression construct or gene expression system. Where the gene expression cassettes, gene expression constructs or gene expression systems comprises a gene encoding a protein of interest, the UTR may be located downstream of said gene. Thus, the UTR may be located between a gene encoding a protein of interest and a terminator sequence.

Most preferably the 3′UTR is immediately downstream of the ORF of the gene (after the stop codon) and upstream of the terminator sequence.

The 3′ UTR may be derived from a bipartite RNA virus, e.g. from the RNA-1 genome segment of a bipartite RNA virus. The UTR may be all or part of the 3′ UTR of the same RNA-1 genome segment from which the enhancer sequence present in the gene expression cassette, gene expression construct or gene expression system is derived, or a variant thereof. Preferably, the UTR is the 3′ UTR of a comoviral RNA-1 genome segment, e.g. the 3′ UTR of the CPMV RNA-1 genome segment.

Thus in another aspect there is provided an expression cassette comprising:
(i) a promoter, operably linked to
(ii) an RNA-1 enhancer sequence;
(iii) a gene of interest it is desired to express;
(iv) a terminator sequence; and optionally
(v) a 3′ UTR located upstream of said terminator sequence.

In another aspect there is provided a gene expression construct comprising:
(a) an RNA-1 enhancer sequence; and
(b) a heterologous sequence for facilitating insertion of a gene encoding a protein of interest into the gene expression system; and optionally
(c) a 3′ UTR.

The heterologous sequence may be a polylinker or multiple cloning site, i.e. a sequence which facilitates cloning of a gene encoding a protein of interest into the expression system. For example, as described hereinafter, the present inventors have provided constructs including a polylinker between the 5′ leader and 3′ UTRs of a CPMV-based expression cassette. Any polylinker may optionally encode one or more sets of Histidine residues to allow the fusion of N— or C terminal His-tags to facilitate protein purification.

The present invention also provides methods of expressing proteins, e.g. heterologous proteins, in host organisms such as plants, yeast, insect or mamalian cells, using a gene expression system of the invention.

Preferred methods are methods of transient expression. As described in the Examples below the system can provide expression levels in relatively short periods of time (3 to 5 days in the Examples).

Methods of the invention may comprise:

(i) use of an expression system, cassette, vector and so on to express a first protein of interest, in conjunction with

(ii) an expression system as described in WO2009/087391 to express a second protein of interest.

The availability of two expression system with different strengths may be beneficial in circumstances where differing levels of expression are desirable e.g. to create complexes or metabolic pathways in which proteins are required in different amounts.

The systems can be used together e.g. sequentially or simultaneously, such that they are present in the same cell at the same time.

Furthermore the availability of construct which differ in their enhancer sequences may be valuable in case of transgenic expression of multiple proteins by the method of Saxena et al. (2011) since the insertion on genes with identical sequences can lead to recombination events. More specifically, Saxena et al. (2011) reports that the CPMV-HT system (described in WO2009/087391) can be used in a stable transgenic as well as a transient format, but that a suppressor of gene silencing such as a mutant form of P19 should be advantageously used with the systems.

Preferably the expression constructs of the invention are present in a vector, and preferably it comprises border sequences which permit the transfer and integration of the expression cassette into the organism genome.

Preferably the construct is a plant binary vector. Preferably the binary transformation vector is based on pPZP (Hajdukiewicz, et al. 1994). Other example constructs include pBin19 (see Frisch, D. A., L. W. Harris-Haller, et al. (1995). “Complete Sequence of the binary vector Bin 19.” Plant Molecular Biology 27: 405-409).

As described herein, the invention may be practiced by moving an expression cassette with the requisite components into an existing pBin expression cassette, or in other embodiments a direct-cloning pBin expression vector may be utilised.

Preferably the vector or other construct further includes a suppressor of gene silencing operably linked to promoter and terminator sequences.

Thus in a further aspect the present invention therefore relates to a gene expression system comprising:

(a) an expression cassette as described above; and

(b) a suppressor of gene silencing operably I inked to promoter and terminator sequences.

The present inventors have shown very high expression levels by incorporating both a gene of interest and a suppressor of silencing onto the same T-DNA as the translational enhance r. Preferred embodiments may therefore utilise all these components are present on the same T-DNA.

However, in an alternative embodiment, the vector or other construct is used in conjunction with a further gene construct encoding the suppressor of gene silencing

Thus, in another aspect the present invention provides a method of expressing a protein in a plant comprising the steps of:

(a) introducing a gene expression construct of the invention into a plant cell; and optionally

(b) introducing a further gene construct comprising a suppressor of gene silencing operably linked to promoter and terminator sequences into the plant cell.

The presence of a suppressor of gene silencing in a gene expression system (including any of those described above) of the invention is preferred but not essential.

The present invention also provides methods comprising introduction of such a construct or constructs into a plant cell.

In a further aspect of the invention, there is disclosed a host cell containing a heterologous construct according to the present invention.

Gene expression vectors of the invention may be transiently or stably incorporated into plant cells.

For small scale production, mechanical agroinfiltration of leaves with constructs of the invention. Scale-up is achieved through, for example, the use of vacuum infiltration.

In other embodiments, an expression vector of the invention may be stably incorporated into the genome of the transgenic plant or plant cell.

In one aspect the invention may further comprise the step of regenerating a plant from a transformed plant cell.

Thus various aspects of the present invention provide a method of transforming a plant cell involving introduction of a construct of the invention into a plant tissue (e.g. a plant cell) and causing or allowing recombination between the vector and the plant cell genome to introduce a nucleic acid according to the present invention into the genome. This may be done so as to effect transient expression i.e. where the vector or construct is introduced into (typically) somatic cells and the protein is generated over a period of time (typically days or weeks) in those cells (see WO01/38512). The cells are not used to regenerate further plants.

Alternatively following transformation of plant tissue, a plant may be regenerated, e.g. from single cells, callus tissue or leaf discs, as is standard in the art.

As described above, the use of the present system in a transgenic context may be preferred if it is desired to create true-breeding lines of plants which can consistently generate large amounts of the desired polypeptide or polypeptides. If multiple genes are to be introduced it may be desirable to minimise repeat sequences. Thus having more than one translation enhancer, each having a different sequence, could be advantageous in avoiding genetic instability and recombination, and avoiding triggering gene silencing.

Regenerated plants or parts thereof may be used to provide clones, seed, selfed or hybrid progeny and descendants (e.g. F1 and F2 descendants), cuttings (e.g. edible parts), propagules, etc.

The invention further provides a transgenic organism (for example obtained or obtainable by a method described herein) in which an expression vector or cassette has been introduced, and wherein the heterologous gene in the cassette is expressed at an enhanced level,

The invention further comprises a method for generating the protein of interest, which method comprises the steps of performing a method (or using an organism) as described above, and optionally harvesting, at least, a tissue in which the protein of interest has been expressed and isolating the protein of interest from the tissue.

Specifically, the present invention therefore provides a transgenic plant or plant cell transiently transfected with an expression vector of the invention.

In a further aspect, the present invention also provides a transgenic plant or plant cell stably transformed with an expression vector of the invention.

The invention also provides a plant propagule from such plants, that is any part which may be used in reproduction or propagation, sexual or asexual, including cuttings, seed and so on. It also provides any part of these plants which includes the plant cells or heterologous DNA described above.

Some particular definitions and embodiments of the invention will now be described in more detail.

Preferred Bipartite viruses for Use in the Present Invention

A “bipartite virus” or virus with a bipartite genome, as referred to herein may be a member of the Comovirinae sub-family of the family Secoviridae. The genera of Comovirinae family include Comovirus, Nepovirus, Fabavirus, Cheravirus and Sadwavirus. Comoviruses include Cowpea mosaic virus (CPMV), Cowpea severe mosaic virus (CPSMV), Squash mosaic virus (SqMV), Red clover mottle virus (RCMV), Bean pod mottle virus (BPMV). Preferably, the bipartite virus (or comovirus) is CPMV.

The sequences of the RNA-1 genome segments of these comoviruses and several specific strains are available from the NCB! database under the accession numbers listed in brackets:

TABLE 1

cowpea mosaic virus RNA-1 (NC_003549)
cowpea severe mosaic virus RNA-1 (NC_003545)
squash mosaic virus RNA-1 strain CH99/211 (EU421059)
squash mosaic virus Japan strain RNA-1 (AB054688)
red clover mottle virus RNA-1 (NC_003741)
bean pod mottle virus RNA-1 (NC_003496)
bean pod mottle virus strain K-Hopkins1 RNA-1 (AF394608)
bean pod mottle virus strain K-Hancock1 RNA-1 (AF394606)
Andean potato mottle virus (M84483 - partial sequence)
Radish mosaic virus Japanese isolate (NC_010709)
Radish mosaic virus California isolate (AB456531)
Broad bean true mosaic virus EV-11 isolate (GU810903)

Other viruses of interest include squash mosaic virus strain Arizona RNA-1.

Numerous sequences from the other genera in the family Comovirinae are also available.

Preferred RNA-1 Enhancer Sequences

“RNA-1 enhancer” sequences (or RNA-1 enhancer elements), as referred to herein, are sequences derived from (or sharing homology with) the RNA-1 genome segment of a bipartite RNA virus, such as a comovirus. Such sequences can enhance downstream expression of a heterologous ORF to which they are attached. Without limitation, it is believed that such sequences (when present in transcribed RNA) can enhance translation of a heterologous ORF to which they are attached.

The enhancer sequence may thus consist or consist essentially of a portion, or fragment, of the RNA-1 genome segment of the bipartite RNA virus from which the RNA-1 enhancer is derived. For example, in one embodiment the nucleic acid does not comprise at least a portion of the coding region of the RNA-1 genome segment from which it is derived. The deleted coding region may be the region of the RNA-1 genome segment encoding the VPg, replicase and protease proteins. In other embodiments the nucleic acid may not comprise any of the original coding region of the RNA-1 genome segment from which it is derived (although it will be understood that the start codon ‘ATG’ following the enhancer sequence would be correspondingly encoded in the RNA-1 genome).

The phrase “consisting essentially of” when used in reference to a nucleic acid, the phrase includes the sequence per se and minor changes and \or extensions that would not affect the enhancer function of the sequence, or provide further (additional) functionality.

As noted above the 5′ UTR of CPMV RNA-1 is 206 nucleotides and the 3′ UTR is 84 nucleotides.

In alternative embodiments of the invention, the RNA-1 enhancer sequence comprises a portion of the sequence of the authentic viral RNA-1 5′ UTR. For example at least 50, 60, 70, 80, 90, 100, 110, 120, 130, 140, 150, 160, 170, 180, 190, 200, 205 contiguous nucleotides thereof.

In other embodiments the RNA-1 enhancer sequence may consist, or consist essentially of between 100 and 206 , more preferably 150 and 200, contiguous nucleotides of the authentic viral RNA-1 5′ UTR.

The portion may start from any nucleotide 1, 2, 3, 4, 5, 6, 7, 8, 9, 10, 20, 30, 40, 50, 60, 70, 80, 90, or 100 nucleotides or more from the 5′ terminus of the authentic viral RNA-1 5′ UTR.

The portion may terminate at any nucleotide 1, 2, 3, 4, 5, 6, 7, 8, 9, 10, 20, 30, 40, 50, 60, 70, 80, 90, or 100 nucleotides or more from the 3′ terminus of the authentic viral RNA-1 5′ UTR.

Non limiting examples of portions would be 1 to 200, 10 to 200, 1 to 150, 5 to 150, 10 to 100, and so on.

Alternative embodiments of the invention are RNA-1 enhancer sequences having at least 99%, 98%, 97%, 96%, 95%, 90%, 85%, 80%, 75%, 70%, 65%, 60%, 55%, or 50% identity to the authentic RNA-1 genome segment or a portion thereof as described above.

Any and all of the above embodiments relating to portions and variants may be applied mutatis mutandis to the 3′ UTR optionally employed in the invention.

Any and all of the above embodiments relating to portions and variants may be applied specifically to the CPMV RNA-1 genome segment shown in the Sequence Annex I.

The terms “percent similarity”, “percent identity” and “percent homology” when referring to a particular Sequence are used as set forth in the University of Wisconsin GCG software program.

RNA-1 enhancer sequences may specifically hybridise with the complementary sequence of the CPMV RNA-1 genome segment sequence shown in the Sequence annex.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. “Complementary” refers to the natural association of nucleic acid sequences by base-pairing (A-G-T pairs with the complementary sequence T-C-A). Complementarity between two single-stranded molecules may be partial, if only some of the nucleic acids pair are complementary; or complete, if all bases pair are complementary. The degree of complementarity affects the efficiency and strength of hybridization and amplification reactions.

Preferred Vectors

“Vector” is defined to include, inter alia, any plasmid, cosmid, phage, viral or Agrobacterium binary vector in double or single stranded linear or circular form which may or may not be self transmissible or mobilizable, and which can transform a prokaryotic or eukaryotic host either by integration into the cellular genome or exist extrachromosomally (e.g. autonomous replicating plasmid with an origin of replication). The constructs used will be wholly or partially synthetic. In particular they are recombinant in that nucleic acid sequences which are not found together in nature (do not run contiguously) have been ligated or otherwise combined artificially. Unless specified otherwise a vector according to the present invention need not include a promoter or other regulatory sequence, particularly if the vector is to be used to introduce the nucleic acid into cells for recombination into the genome.

“Binary Vector”: as is well known to those skilled in the art, a binary vector system includes (a) border sequences which permit the transfer of a desired nucleotide sequence into a plant cell genome; (b) desired nucleotide sequence itself, which will generally comprise an expression cassette of (i) a plant active promoter, operably linked to (ii) the target sequence and\or enhancer as appropriate. The desired nucleotide sequence is situated between the border sequences and is capable of being inserted into a plant genome under appropriate conditions. The binary vector system will generally require other sequence (derived from A. tumefaciens) to effect the integration. Generally this may be achieved by use of so called “agro-infiltration” which uses Agrobacterium-mediated transient transformation. Briefly, this technique is based on the property of Agrobacterium tumefaciens to transfer a portion of its DNA (“T-DNA”) into a host cell where it may become integrated into nuclear DNA. The T-DNA is defined by left and right border sequences which are around 21-23 nucleotides in length. The infiltration may be achieved e.g. by syringe (in leaves) or vacuum (whole plants). In the present invention the border sequences will generally be included around the desired nucleotide sequence (the T-DNA) with the one or more vectors being introduced into the plant material by agro-infiltration.

Preferred vectors are based on improvements to the pBINPLUS vector whereby it has been shown that it is possible to drastically reduce the size of the vector without compromising performance in terms of replication and TDNA transfer. Furthermore, elements of the enhancer system (as exemplified by the so-called “CP MV-HT” and “CPMV-RT” systems) have been incorporated into the resulting vector in a modular fashion such that multiple proteins can be expressed from a single T-DNA. These improvements have led to the creation of a versatile, high-level expression vector that allows efficient direct cloning of foreign genes.

These examples represent preferred binary plant vectors. Preferably they include the ColEI origin of replication, although plasmids containing other replication origins that also yield high copy numbers (such as pRi-based plasmids, Lee and Gelvin, 2008) may also be preferred, especially for transient expression systems.

If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

Most preferred vectors are the pEAQ vectors described below which permit direct cloning version by use of a polylinker between the 5′ leader and 3′ UTRs of an expression cassette including a translational enh ancer of the invention, positioned on a T-DNA which also contains a suppressor of gene silencing (“p19”) and an NPTII cassettes.

An advantage of pEAQ-derived vectors is that each component of a multi-chain protein such as an IgG can automatically be delivered to each infected cell.

Preferred Suppressors of Gene Silencing

Suppressors of gene silencing useful in these aspects are known in the art and described in WO/2007/135480. They include HcPro from Potato virus Y, He-Pro from TEV, P19 from TBSV, rgsCam, B2 protein from FHV, the small coat protein of CPMV, and coat protein from TCV.

A preferred suppressor when producing stable transgenic plants is the P19 suppressor incorporating a R43W mutation.

Nucleic Acids

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to RNA, refers to a RNA molecule that is separated from sequences with which it is immediately contiguous in the naturally occurring genome of the organism in which it originated.

For example, an “isolated nucleic acid” may comprise a nucleic acid molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

Promoter

A “promoter” is a sequence of nucleotides from which transcription may be initiated of DNA operably linked downstream (i.e. in the 3′ direction on the sense strand of double-stranded DNA).

In the present invention the promoter will generally not be a promoter recognised by the T7 polymerase.

“Operably linked” means joined as part of the same nucleic acid molecule, suitably positioned and oriented for transcription to be initiated from the promoter.

Preferably the promoter used to drive the gene of interest will be a plant promoter. Preferably it will be a “strong” promoter. Examples of strong promoters for use in plants include:

(1) p35S: Odell et al., 1985

(2) Cassava Vein Mosaic Virus promoter, pCAS, Verdaguer et al., 1996

(3) Promoter of the small subunit of ribulose biphosphate carboxylase, pRbcS:

Outchkourov et al., 2003.

Other strong promoters include pUbi (for monocots and dicots) and pActin.

The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

Terminator

The termination (terminator) sequence may be a termination sequence derived from the RNA-1 genome segment of a bipartite RNA virus, e.g. a comovirus. In one embodiment the termination sequence may be derived from the same bipartite RNA virus from which the enhancer sequence is derived. The termination sequence may comprise a stop codon. Termination sequence may also be followed by polyadenylation signals.

Expression Cassette

“Expression cassette” refers to a situation in which a nucleic acid is under the control of, and operably linked to, an appropriate promoter or other regulatory elements for transcription in a host cell such as a microbial or plant cell.

Plant Transformation

Specific procedures and vectors previously used with wide success upon plants are described by Guerineau and Mullineaux (1993) (Plant transformation and expression vectors. In: Plant Molecular Biology Labfax (Croy R R D ed) Oxford, BIOS Scientific Publishers, pp 121-148). Suitable vectors may include plant viral-derived vectors (see e.g. EP-A-194809). If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

Nucleic acid can be introduced into plant cells using any suitable technology, such as a disarmed Ti-plasmid vector carried by Agrobacterium exploiting its natural gene transfer ability (EP-A-270355, EP-A-0116718, NAR 12(22) 8711 - 87215 1984; the floral dip method of Clough and Bent, 1998), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d) Physical methods for the transformation of plant cells are reviewed in Oard, 1991, Biotech. Adv. 9: 1-11. Ti-plasmids, particularly binary vectors, are discussed in more detail below.

Agrobacterium transformation is widely used by those skilled in the art to transform dicotyledonous species. However there has also been considerable success in the routine production of stable, fertile transgenic plants in almost all economically relevant monocot plants (see e.g. Hiei et al. (1994) The Plant Journal 6, 271-282)).

Microprojectile bombardment, electroporation and direct DNA uptake are preferred where Agrobacterium aloneis inefficient or ineffective. Alternatively, a combination of different techniques may be employed to enhance the efficiency of the transformation process, e.g. bombardment with Agrobacterium coated microparticles (EP-A-486234) or microprojectile bombardment to induce wounding followed by co-cultivation with Agrobacterium (EP-A-486233).

The particular choice of a transformation technology will be determined by its efficiency to transform certain plant species as well as the experience and preference of the person practising the invention with a particular methodology of choice.

It will be apparent to the skilled person that the particular choice of a transformation system to introduce nucleic acid into plant cells is not essential to or a limitation of the invention, nor is the choice of technique for plant regeneration. In experiments performed by the inventors, the enhanced expression effect is seen in a variety of integration patterns of the T-DNA.

Following transformation of plant tissue, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The generation of fertile transgenic plants has been achieved in the cereals such as rice, maize, wheat, oat, and barley plus many other plant species (reviewed in Shimamoto, K. (1994) Current Opinion in Biotechnology 5, 158-162.; Vasil, et al. (1992) Bio/Technology 10, 667-674; Vain et al., 1995, Biotechnology Advances 13 (4): 653-671; Vasil, 1996, Nature Biotechnology 14 page 702).

Genes and Sequences of Interest

“Gene” unless context demands otherwise refers to any nucleic acid encoding genetic information for translation into a peptide, polypeptide or protein. Thus unless context demands otherwise it used interchangeably with “ORF”.

The genes which it may be desired to express may be transgenes or endogenes (in respect of the host in which the systems are employed).

In one embodiment the protein may be one that is unstable or is toxic. In this embodiment the rapid kinetics of the systems described herein may be advantageous.

As described herein, the protein may be expressed in conjunction with other proteins in the same cell e.g. to create complexes, metabolic pathways, or assemble multimers in a defined fashion.

Genes of interest include those encoding agronomic traits, insect resistance, disease resistance, herbicide resistance, sterility, grain characteristics, and the like. The genes may be involved in metabolism of oil, starch, carbohydrates, nutrients, etc. Thus genes or traits of interest include, but are not limited to, environmental- or stress-related traits, disease-related traits, and traits affecting agronomic performance. Target sequences also include genes responsible for the synthesis of proteins, peptides, fatty acids, lipids, waxes, oils, starches, sugars, carbohydrates, flavors, odors, toxins, carotenoids, hormones, polymers, flavonoids, storage proteins, phenolic acids, alkaloids, lignins, tannins, celluloses, glycoproteins, glycolipids, etc.

Most preferably the targeted genes in monocots and/or dicots may include those encoding enzymes responsible for oil production in plants such as rape, sunflower, soya bean and maize; enzymes involved in starch synthesis in plants such as potato, maize, cereals; enzymes which synthesise, or proteins which are themselves, natural medicaments such as pharmaceuticals or veterinary products.

Heterologous nucleic acids may encode, inter alia, genes of bacterial, fungal, plant or animal origin. The polypeptides may be utilised in planta (to modify the characteristics of the plant e.g. with respect to pest susceptibility, vigour, tissue differentiation, fertility, nutritional value etc.) or the plant may be an intermediate for producing the polypeptides which can be purified therefrom for use elsewhere. Such proteins include, but are not limited to retinoblastoma protein, p53, angiostatin, and leptin. Likewise, the methods of the invention can be used to produce mammalian regulatory proteins. Other sequences of interest include proteins, hormones, growth factors, cytokines, serum albumin, haemoglobin, collagen, etc.

Thus the target gene or nucleotide sequence preferably encodes a protein of interest which is: an insect resistance protein; a disease resistance protein; a herbicide resistance protein; a mammalian protein.

Plants

Plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum)), proso millet (Panicum miliaceum), foxtail millet (Setaria italica), finger millet, (Eleusine coracana)), sunflower (Helianthus annuus), safflower (Carthamus tinctorius), wheat (Triticum aestivum), soybean (Glycine max), tobacco (Nicotiana tabacum), Nicotiana benthamiana, potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1a. Map of the vector created for expression of genes with UTRs of CPMV RNA-1.

FIG. 1b. Vector map of the construct generated for expression of GFP with UTRs of CPMV RNA-1.

FIG. 2a. Expression levels of RT-GFP based on spectrofluorometry from tissue harvested over a period of 12 days. Each bar represents GFP expressed in grams per kilogram of fresh weight tissue (FWT). Error bars represent standard deviation between biological replicates.

FIG. 2b. Proteins from leaf tissue infiltrated with RT-GFP separated and analysed by SDS-PAGE using a 12% polyacrylamide gel. An extract from a plant infiltrated with empty vector was used as a negative control (-). 500 ng of recombinant GFP was used as the positive (+).

FIG. 3. Vector map of the construct used for expression of GFP in the CPMV RNA-2 based HT system

FIG. 4a. Expression levels of RT-GFP and HT-GFP based on spectrofluorometry from tissue harvested over a period of 12 days. Each bar represents GFP expressed in grams per kilogram of fresh weight tissue (FWT). Error bars represent standard deviation between biological replicates.

FIG. 4b. Proteins from leaf tissue infiltrated with RT-GFP and HT-GFP separated and analysed by SDS-PAGE using a 12% polyacrylamide gel. An extract from a plant infiltrated with empty vector was used as the negative control (−). 500 ng of recombinant GFP was used as the positive control (+).

FIG. 5a. Expression of GFP from RT and HT constructs visualised under ultraviolet light 6 dpi

FIG. 5b. Expression of GFP from RT and HT constructs visualised under ultraviolet light 9 dpi

FIG. 5c. Expression of GFP from RT and HT constructs visualised under ultraviolet light 12 dpi

### SEQUENCES

I) The complete CPMV RNA-1 genome segment (nucleotides 1 to 5889)

II) Sequence of RNA-1 UTRs used in this study

III) Vector NTI format description of pEAQexpress-RT-GFP

### EXAMPLES

### Example 1

### Methods

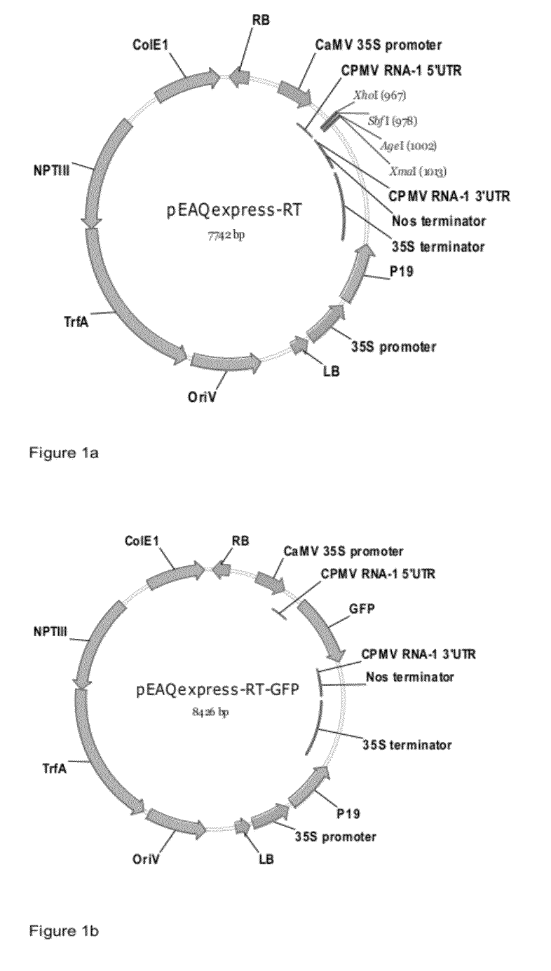

The pEAQ binary vectors for plant expression (Sainsbury et al., 2009) were modified to encode the UTRs of CPMV RNA-1, in place of the UTRs from RNA-2, to create a construct called pEAQexpress-RT (FIG. 1a). pEAQexpress-RT contains a polylinker to enable introduction of a gene of interest between the RNA-1 UTRs. In this study, the gene of interest was gfp which encodes the green fluorescent protein (GFP). gfp was cloned into the polylinker to generate pEAQexpress-RT-GFP, in which gfp is flanked by the 5′ UTR and the 3′ UTR of CPMV RNA-1 (FIG. 1b).

Manipulation of the pEAQ constructs was undertaken using standard restriction enzyme-based cloning methods in Escherischia coli TOP10 cells (Invitrogen). Once verified by sequencing, pEAQexpress-RT-GFP was transformed into Agrobacterium tumefaciens strain LBA4404. Transformed Agrobacterium suspensions were infiltrated into young fully expanded leaves of 3-week old Nicotiana benthamiana plants using the technique of syringe infiltration. Leaves were harvested from 1 to 12 days post infiltration (dpi) and analysed for GFP expression levels. GFP expression was monitored using a 100 W handheld long-wave ultraviolet (UV) lamp and quantified by spectrofluorometry using a SPECTRAmax spectrofluorometer (Molecular Devices). Each measurement was done in triplicate and averaged. In addition, for each time point, three biological replicates were used.

### Results

GFP Expression Levels in the CPMV-RT System

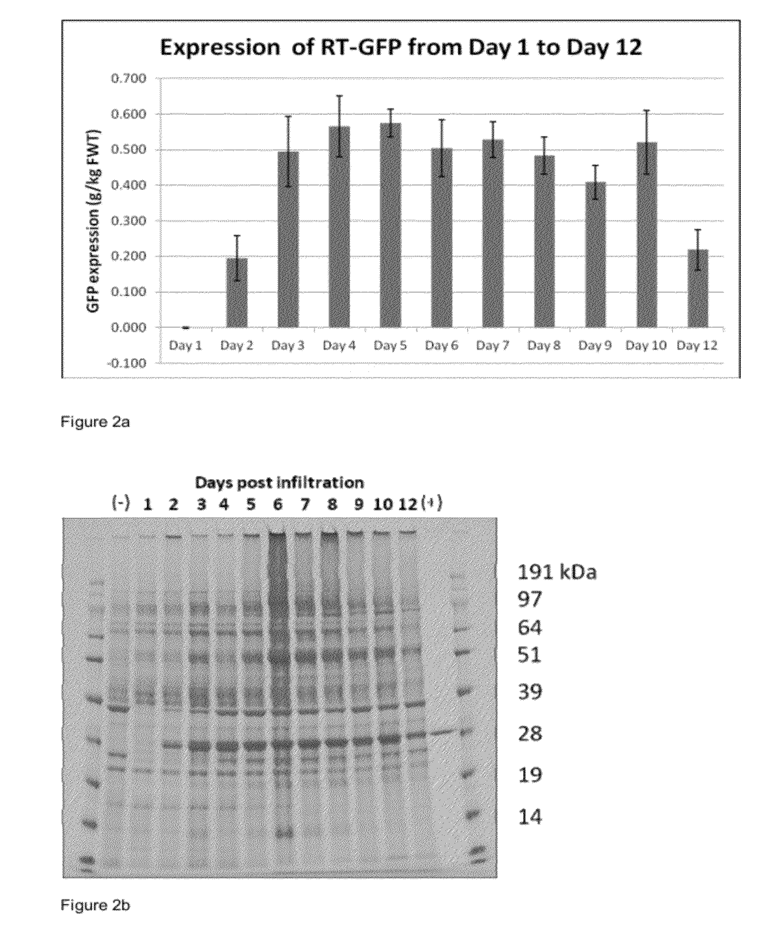

Expression of RT-GFP was monitored from 1 to 12 dpi. For each time point, leaves were harvested and from the crude extract, GFP fluorescence was measured (FIG. 2a). In FIG. 2A, every bar represents grams of GFP expressed per kilogram of fresh weight tissue (FWT). Values are averages from expression levels of three biological replicates and error bars represent standard deviation. Furthermore, crude extracts were analysed on 12% polyacrylamide gels by standard SDS-PAGE and the intensity of bands for GFP were compared (FIG. 2b). RT-GFP is expressed to levels of 0.5-0.6 g/kg FWT with maximal expression 4-5 days after infiltration (dpi).

Comparison of the CPMV-RT with CPMV-HT System

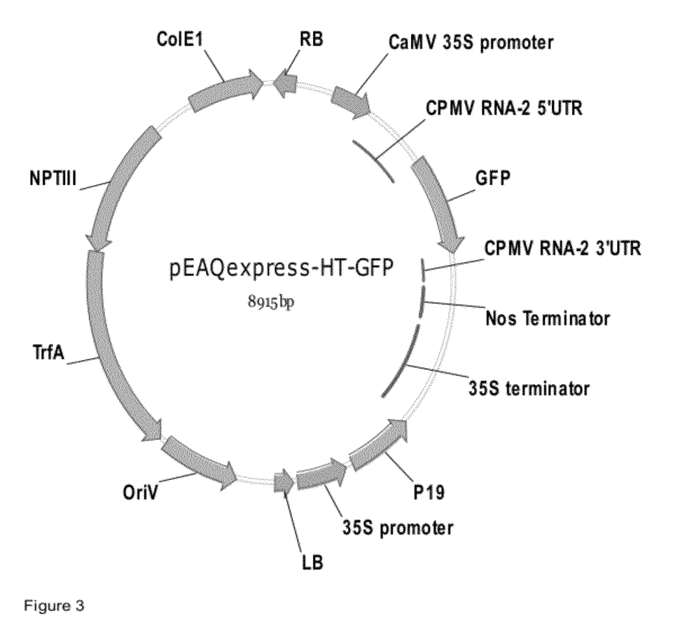

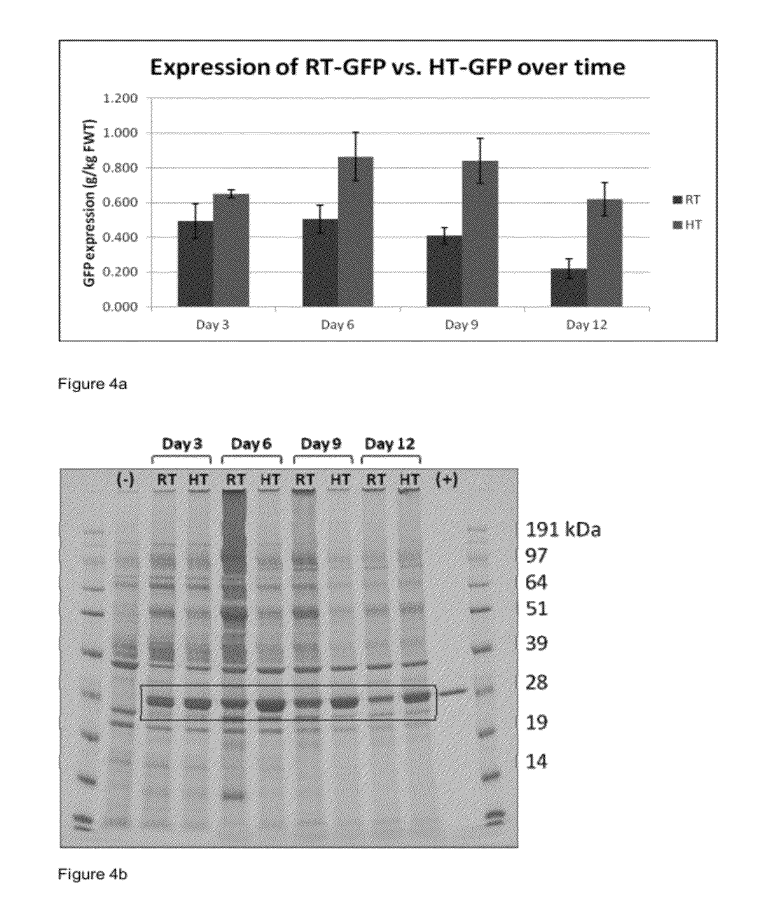

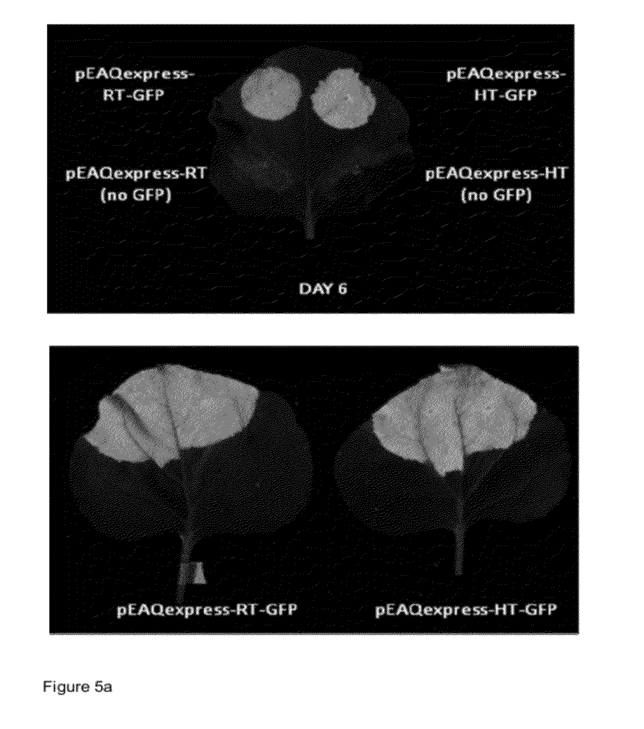

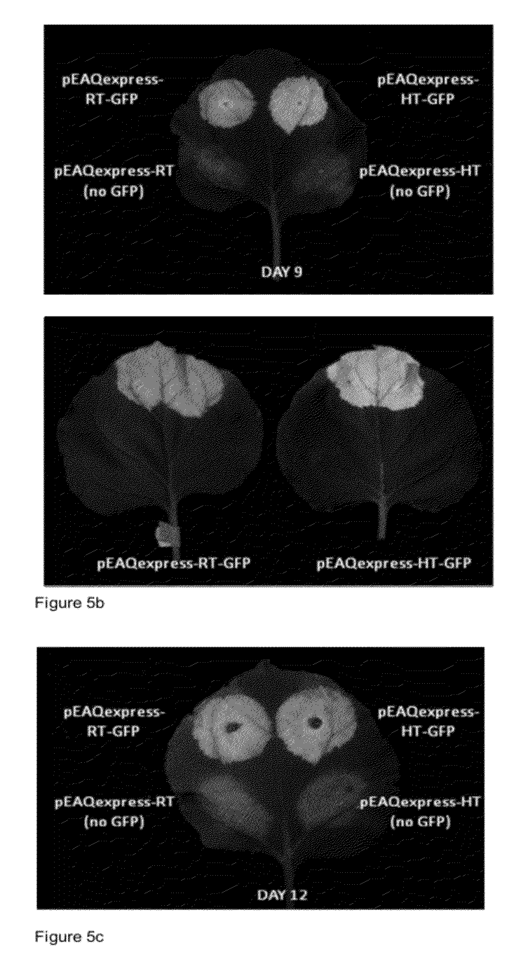

A vector based on the CPMV-HT system, pEAQexpress-HT-GFP (FIG. 3), was tested in parallel with pEAQ-RT-GFP to compare and contrast the two expression systems. Expression of GFP was monitored 3, 6, 9 and 12 dpi by fluorescence measurements (FIG. 4a) and analysis of the proteins present in the crude extract using 12% polyacrylamide gels (FIG. 4b). In addition, leaves were photographed under ultraviolet light to visualise GFP expression (FIG. 5). As controls, infiltrations with Agrobacterium harbouring empty vectors (no gene cloned in between the UTRs) were done.

### CONCLUSIONS

The results showed that high levels of GFP could be expressed in pEAQ constructs in which the modified 5′ UTR and 3′ UTR from RNA-2 were replaced by the 5′ and 3′ UTRs from RNA-1 (CPMV-RT). The maximum expression level was found to occur between days 3-5 after infiltration after which expression declined (FIGS. 2a and 2b), and this maximum was seemingly earlier than with the CPMV-HT system (FIG. 4a). Thus the kinetics of expression differ between CP MV-RT and CPMV-HT.

The rapid rise in expression seen with CPMV-RT could be particularly beneficial to achieve expression of a protein that is unstable or has toxic effects on the plant. The availability of two expression system with different strengths may be beneficial in circumstances where differing levels of expression are desirable to create complexes or metabolic pathways in which proteins are required in different amounts. Finally, the availability of construct which differ in sequence in the UTRs may be valuable in case of transgenic expression of multiple proteins by the method of Saxena et al. (2011) since the insertion on genes with identical sequences can lead to recombination events.

SEQUENCE ANNEX I

COMPLETE CPMV RNA-1 GENOME SEGMENT (5889 bp)

| | |
|---|---|
| 1 | tattaaaatc aatacaggtt ttgataaaag cgaacgtgga gaaatccaaa cctttctttc |
| 61 | tttcctcaat ctcttcaatt gcgaacgaaa tccaagcttt ggttttgctg aaacaaatac |
| 121 | acaacgtata ctgaatttgg caaatttctc tctctctctc tgtcattttc tttcttctgt |
| 181 | cgggactttc ttagtcttga cccaacatgg gtctcccaga atatgaggcc gatagtgagg |
| 241 | ctttattaag tcaactcact atcgaattca cacccggcat gacagtttct tcattgttgg |
| 301 | cacaagtcac cactaatgac tttcacagtg ccattgagtt ttttgctgca gaaaaagcag |
| 361 | tagacattga gggcgttcat tacaatgcgt atatgcaaca aattaggaaa aaccctagtt |
| 421 | tattacgcat ttccgtggta gcttatgctt tccacgtttc agacatggta gctgagacca |
| 481 | tgtcttatga tgtttatgaa tttctgtata aacattatgc ccttttcatc tctaatctgg |
| 541 | tgaccagaac actcagattt aaagagcttt tgctgttctg taagcagcaa tttctggaga |
| 601 | aaatgcaagc ttcaatagtc tgggctccgg aacttgagca atatcttcaa gttgaagggg |
| 661 | atgctgtggc tcaaggagtt tcacaactgt tatacaagat ggtcacttgg gtgcccactt |
| 721 | ttgtcagagg agcagtagac tggagcgttg atgcgatttt ggtcagtttc aggaaacatt |
| 781 | ttgaaaagat ggttcaggag tatgtgccca tggctcatcg cgtttgcagt tggctgagcc |
| 841 | aactatggga taagatcgtg caatggatct cacaagcaag tgagaccatg ggttggtttc |
| 901 | tagatggttg tcgggatttg atgacttggg gaattgccac tctcgcaaca tgtagtgctc |
| 961 | tctccctggt tgagaagctg ttagtcgcaa tgggttttct ggttgagcct ttcggcttga |
| 1021 | gtggaatctt cttgcggacg ggagttgttg cggcagcttg ttataactat gggactaatt |
| 1081 | ctaagggttt tgccgagatg atggctttgt tgtcattggc ggctaactgt gtctctacag |
| 1141 | ttatagttgg tggctttttc cctggtgaaa aggacaatgc acagagtagt cctgttatcc |
| 1201 | tcttagaagg attggctggg cagatgcaaa acttttgtga gactacactt gtcagtgttg |
| 1261 | ggaaaacatg cactgccgtc aatgctatct caacatgttg tgggaatctg aaagcactgg |
| 1321 | ccggaaggat cttgggcatg ctcagagatt ttatctggaa gactttgggc tttgagacca |
| 1381 | gatttctagc agatgcatct ttgctttttg gcgaggatgt tgatggatgg ctcaaagcaa |
| 1441 | tcagtgatct gcgagatcaa tttattgcca aatcatactg ttcgcaggat gagatgatgc |
| 1501 | agattttggt gttgcttgaa aagggaaggc agatgcggaa aagtggtctt tctaaaggag |
| 1561 | gcatttctcc tgctatcatt aatctgattc tcaaagggat taatgatctt gaacaattga |
| 1621 | accgcagctg ttcagtgcaa ggagtaagag gagttaggaa aatgccattt accattttct |
| 1681 | tccaaggaaa gtcacgcact ggtaagagtt tgctgatgag tcaggttaca aaggattttc |
| 1741 | aggatcacta tggattgggt ggagaaactg tgtacagtag aaatccttgt gatcaatatt |
| 1801 | ggagtggata tcggcggcaa ccttttgtgc tgatggatga ttttgccgcc gttgttactg |
| 1861 | agccgtctgc tgaggctcag atgatcaatc tgatttctag tgctccatat cctttgaata |
| 1921 | tggctggact tgaagaaaaa ggaatttgtt ttgattctca atttgttttt gtttccacca |
| 1981 | acttcttgga agtatctcct gaagccaaag ttagggacga tgaggctttc aagaacagga |
| 2041 | gacatgtgat tgttcaggtt tcaaatgatc ctgccaaagc atatgatgct gcaaattttg |
| 2101 | ctagcaacca aatttacacc attttggcat ggaaggatgg tcgatacaac accgtgtgcg |
| 2161 | ttattgagga ctatgatgag ctggtggcat atttgttgac taggagtcaa cagcatgctg |
| 2221 | aagagcagga gaagaatctt gctaacatga tgaagagtgc tacatttgaa agtcatttca |
| 2281 | aaagtttagt tgaagtcctt gagctcggtt ctatgatatc tgctggtttt gatatcattc |
| 2341 | ggccagaaaa acttcctagt gaagctaagg agaagagagt cctttacagt attccctaca |
| 2401 | atggggagta ttgtaatgca ctcattgatg acaattacaa tgttacttgc tggtttggtg |
| 2461 | agtgtgttgg taatcctgag cagctctcta agtacagtga aaagatgctt ttgggtgctt |
| 2521 | atgaatttct tctgtgttct gagagcttga atgttgtaat tcaggcacat ttgaaggaaa |
| 2581 | tggtttgccc tcaccattat gacaaggagc tcaattttat tggcaagata ggagagacct |
| 2641 | actatcacaa tcagatggtt tcaaatatcg gctctatgca gaaatggcat cgtgccattc |
| 2701 | tgtttggaat tggggttctc ttgggaaagg aaaaagagaa gacatggtac caagttcagg |
| 2761 | ttgccaatgt taaacaagct ctttacgaca tgtacactaa ggagattcgt gattggccca |
| 2821 | tgccgatcaa agtcacctgt ggaattgtct tggcagctat tgggggtagt gccttttgga |
| 2881 | aagtgtttca acaactagtg ggaagcggaa atggtccagt attgatgggt gtggctgctg |
| 2941 | gagcattcag tgctgagcct caaagtagaa agcccaatag gtttgatatg cagcaataca |
| 3001 | ggtacaacaa tgttcctctc aagagaagag tttgggcaga cgcacaaatg tctttggatc |
| 3061 | agagtagtgt tgctatcatg tctaagtgta gggctaatct ggtttttgga ggcactaatt |
| 3121 | tgcaaatagt catggtacca ggaagacgct ttttggcatg caaacatttc ttcacccaca |
| 3181 | taaagaccaa attgcgtgtg gaaatagtta tggatggaag aaggtactat catcaatttg |
| 3241 | atcctgcaaa tatttatgat atacctgatt ctgagttggt cttgtactcc catcctagct |
| 3301 | tggaagacgt ttcccattct tgctgggatc tgttctgttg ggacccagac aaagaattgc |
| 3361 | cttcagtatt tggagcggat ttcttgagtt gtaaatacaa caagtttggg ggtttttatg |
| 3421 | aggcgcaata tgctgatatc aaagtgcgca caaagaaaga atgccttacc atacagagtg |
| 3481 | gtaattatgt gaacaaggtg tctcgctatc ttgagtatga agctcctact atccctgagg |
| 3541 | attgtggatc tcttgtgata gcacacattg gtgggaagca caagattgtg ggtgttcatg |
| 3601 | ttgctggtat tcaaggtaag ataggatgtg cttccttatt gccaccattg gagccaatag |
| 3661 | cacaagcgca aggtgctgag gaatactttg attttcttcc agctgaagag aatgtatctt |
| 3721 | ctggagtggc tatggtagca ggactcaaac aaggagttta cataccatta cccacaaaaa |
| 3781 | cagcgctagt ggagaccccc tccgagtggc atttggacac accatgtgac aaagttccta |
| 3841 | gcattttagt tcccacggat ccccgaattc ctgcgcaaca tgaaggatat gatcctgcta |
| 3901 | agagtggggt ttccaagtat tcccagccta tgtctgctct ggaccctgag ttacttggcg |
| 3961 | aggtggctaa tgatgttctc gagctatggc atgactgcgc tgtagattgg gacgattttg |
| 4021 | gtgaagtgtc tctggaggaa gctttgaatg gatgtgaagg agtggaatat atggaaagga |
| 4081 | ttccattagc aacttctgag ggctttccgc acattctttc tagaaatggg aaagaaaagg |
| 4141 | ggaaaagacg gtttgttcag ggagatgatt gtgttgtctc actaattcca ggaactactg |
| 4201 | tagccaaagc ttatgaggag ttggaagcaa gtgcacacag atttgttccc gctcttgttg |
| 4261 | ggattgaatg tccaaaagat gagaagttgc ctatgagaaa ggtttttgat aagcctaaga |
| 4321 | ccaggtgttt taccattttg ccaatggaat ataatttggt cgttcgtagg aagtttctga |
| 4381 | attttgtgcg ctttatcatg gccaatcgtc acagactcag ttgtcaagtg ggtattaatc |
| 4441 | catattcaat ggaatggagt cgcttagcag caaggatgaa agagaaaggc aatgatgtct |
| 4501 | tgtgttgtga ttatagctca ttcgatggct tgctttctaa gcaagtgatg gatgtcattg |
| 4561 | ctagcatgat caatgaactt tgtggtggag aggatcaact caaaaatgca aggcgaaact |
| 4621 | tgttaatggc gtgttgctct aggttggcta tttgcaagaa tacagtatgg agagttgagt |
| 4681 | gtggtattcc ttcagggttt ccaatgacag tgattgtgaa tagcattttt aatgagattc |
| 4741 | tcattcgcta tcattacaag aaactcatgc gcgaacaaca agctcctgaa ctgatggtac |
| 4801 | agagttttga taaactcata gggctggtga cttatggtga tgataatctg atttcagtga |
| 4861 | atgctgttgt gacaccctat tttgatggga agaaattgaa gcaatctttg gctcagggtg |
| 4921 | gtgtgactat cactgatggt aaggacaaaa caagtttgga acttcctttt cgcagattgg |
| 4981 | aagaatgtga ttttctcaag agaacttttg ttcagaggag cagtaccatc tgggacgctc |
| 5041 | cagaggataa ggcaagtttg tggtcgcagc ttcattatgt taattgcaac aattgtgaga |
| 5101 | aagaagttgc ttatttgact aatgttgtta atgttcttcg tgaactttat atgcatagtc |
| 5161 | ctcgggaagc cacagaattt aggaggaagg tcttaaagaa ggtcagttgg atcactagtg |
| 5221 | gagatttgcc tactttggca caattgcaag agttctatga gtaccagcgg cagcaaggtg |
| 5281 | gggcagacaa caatgacact tgtgacttgt taacaagtgt agacttgcta ggtcctcctt |
| 5341 | tgtcttttga gaaagaagcg atgcacggat gcaaagtgtc tgaagaaatc gtcaccaaga |
| 5401 | atttggcata ttacgatttc aaaaggaaag gtgaggatga agtggtattt ctgttcaata |
| 5461 | cgctctatcc tcagagttca ttgcctgatg ggtgtcactc tgtgacctgg tctcagggta |
| 5521 | gtggaagggg aggtttgccc acacaaagtt ggatgagcta taatataagc aggaaagatt |
| 5581 | ctaatatcaa caagattatt agaactgctg tttcttcgaa gaaacgagtg atattctgtg |
| 5641 | ctcgtgataa tatggttcct gttaacattg tagctttgct ctgtgctgtt agaaacaagc |
| 5701 | tgatgcccac tgctgtatct aatgctacac ttgtcaaggt gatggaaaat gccaaagctt |
| 5761 | tcaagttttt accagaagag ttcaatttcg ctttttctga tgtttaggta aataatgctt |
| 5821 | atgtttttgt ttgctcctgt ttagcaggtc gttccttcag caagaacaac aaaaatatgt |
| 5881 | gtttttatt |

The 5′ and 3′ UTR regions are shown in bold and underlined.

SEQUENCE ANNEX II

THE RT EXPRESSION CASSETTE (908 bp)
Includes:
CaMV promoter 1-315 (315)
CPMV RNA-1 5′UTR 316-521 (206)
Polylinker 522-573 (52)
CPMV RNA-1 3′UTR 574-655 (82)
Nos terminator 656-908 (253)
(CPMV Sequences obtained from GenBank accession
no. NC_003549).

| | |
|---|---|
| 1 | GGAAACCTCC TCGGATTCCA TTGCCCAGCT ATCTGTCACT |
| 41 | TTATTGAGAA GATAGTGGAA AAGGAAGGTG GCTCCTACAA |
| 81 | ATGCCATCAT TGCGATAAAG GAAAGGCCAT CGTTGAAGAT |
| 121 | GCCTCTGCCG ACAGTGGTCC CAAAGATGGA CCCCCACCCA |
| 161 | CGAGGAGCAT CGTGGAAAAA GAAGACGTTC CAACCACGTC |
| 201 | TTCAAAGCAA GTGGATTGAT GTGATATCTC CACTGACGTA |
| 241 | AGGGATGACG CACAATCCCA CTATCCTTCG CAAGACCCTT |
| 281 | CCTCTATATA AGGAAGTTCA TTTCATTTGG AGAGGTATTA |
| 321 | AAATCAATAC AGGTTTTGAT AAAAGCGAAC GTGGAGAAAT |
| 361 | CCAAACCTTT CTTTCTTTCC TCAATCTCTT CAATTGCGAA |
| 401 | CGAAATCCAA GCTTTGGTTT TGCTGAAACA AATACACAAC |
| 441 | GTATACTGAA TTTGGCAAAT TTCTCTCTCT CTCTCTGTCA |
| 481 | TTTTCTTTCT TCTGTCGGGA CTTTCTTAGT CTTGACCCAA |
| 521 | CCCTCGAGCC TGCAGGCAAT TGTATACCTA GGTCCGGACC |
| 561 | GGTACGTACC CGGGTAAATA ATGCTTATGT TTTTGTTTGC |
| 601 | TCCTGTTTAG CAGGTCGTTC CTTCAGCAAG AACAACAAAA |
| 641 | ATATGTGTTT TTATTGATCG TTCAAACATT TGGCAATAAA |
| 681 | GTTTCTTAAG ATTGAATCCT GTTGCCGGTC TTGCGATGAT |
| 721 | TATCATATAA TTTCTGTTGA ATTACGTTAA GCATGTAATA |
| 761 | ATTAACATGT AATGCATGAC GTTATTTATG AGATGGGTTT |
| 801 | TTATGATTAG AGTCCCGCAA TTATACATTT AATACGCGAT |
| 841 | AGAAAACAAA ATATAGCGCG CAAACTAGGA TAAATTATCG |
| 881 | CGCGCGGTGT CATCTATGTT ACTAGATC |

SEQUENCE ANNEX III

```
LOCUS pEAQexpress-RT-G 8426 by DNA circular
SOURCE
  ORGANISM
COMMENT This file is created by Vector NTI
        http://www.invitrogen.com/
COMMENT VNTDATE|579018716|
COMMENT VNTDBDATE|579977961|
COMMENT LSOWNER|
COMMENT VNTNAME|pEAQexpress-RT-GFP|
COMMENT VNTAUTHORNAME|Pooja|
COMMENT VNTAUTHOREML|pooja.saxena@bbsrc.ac.uk|
FEATURES Location/Qualifiers
    CDS 978 . . . 1697
            /vntifkey = “4”
            /label = GFP
            /note = “gene encoding green fluorescent protein from pEAQ-HT-GFP”
    terminator 1787 . . . 2039
            /vntifkey = “43”
            /label = Nos\terminator
            /note = “Nopaline synthase terminator”
    3′UTR 1705 . . . 1786
            /vntifkey = “50”
            /label = CPMV\RNA-1\3′UTR
            /note = “3′ UTR of CPMV RNA-1”
    5′UTR 763 . . . 968
            /vntifkey = “52”
            /label = CPMV\RNA-1\5′UTR
            /note = “5′ UTR of CPMV RNA-1”
    promoter 448 . . . 762
            /vntifkey = “29”
            /label = CaMV\35S\promoter
            /note = “CaMV 35S promoter”
    promoter complement(3387 . . . 3786)
            /vntifkey = “29”
            /label = 35S\promoter
    CDS complement(2815 . . . 3333)
            /vntifkey = “4”
            /label = P19
    terminator complement(2109 . . . 2808)
            /vntifkey = “43”
            /label = 35S\terminator
    CDS complement(4945 . . . 6426)
            /vntifkey = “4”
            /label = TrfA
    rep_origin complement(4273 . .. 4890)
            /vntifkey = “33”
            /label = OriV
    misc_recomb complement(8424 . . . 159)
            /vntifkey = “86”
            /label = RB
    misc_recomb complement(3821 . . . 3968)
            /vntifkey = “86”
            /label = LB
    rep_origin 7753 . . . 8342
            /vntifkey = “33”
            /label = ColE1
            /note = “opposite orientation to what's annotated in published pBINplus
map”
    CDS complement(6427 . . . 7419)
            /vntifkey = “4”
            /label = NPTIII
BASE COUNT 2029 a 2186 c 2006 g 2205 t
ORIGIN
```

| | |
|---|---|
| 1 | gtggttggca tgcacataca aatggacgaa cggataaacc ttttcacgcc cttttaaata |
| 61 | tccgattatt ctaataaacg ctcttttctc ttaggtttac ccgccaatat atcctgtcaa |
| 121 | acactgatag tttgtgaacc atcacccaaa tcaagttttt tggggtcgag gtgccgtaaa |
| 181 | gcactaaatc ggaaccctaa agggagcccc cgatttagag cttgacgggg aaagccggcg |
| 241 | aacgtggcga gaaaggaagg gaagaaagcg aaaggagcgg gcgccattca ggctgcgcaa |
| 301 | ctgttgggaa gggcgatcgg tgcgggcctc ttcgctatta cgccagctgg cgaaaggggg |
| 361 | atgtgctgca aggcgattaa gttgggtaac gccagggttt tcccagtcac gacgttgtaa |
| 421 | aacgacggcc agtgaattgt taattaagga aacctcctcg gattccattg cccagctatc |
| 481 | tgtcacttta ttgagaagat agtggaaaag gaaggtggct cctacaaatg ccatcattgc |
| 541 | gataaaggaa aggccatcgt tgaagatgcc tctgccgaca gtggtcccaa agatggaccc |
| 601 | ccacccacga ggagcatcgt ggaaaaagaa gacgttccaa ccacgtcttc aaagcaagtg |
| 661 | gattgatgtg atatctccac tgacgtaagg gatgacgcac aatcccacta tccttcgcaa |
| 721 | gacccttcct ctatataagg aagttcattt catttggaga ggtattaaaa tcaatacagg |
| 781 | ttttgataaa agcgaacgtg gagaaatcca aacctttctt tctttcctca atctcttcaa |
| 841 | ttgcgaacga aatccaagct ttggttttgc tgaaacaaat acacaacgta tactgaattt |
| 901 | ggcaaatttc tctctctctc tctgtcattt tctttcttct gtcgggactt tcttagtctt |
| 961 | gacccaaccc tcgagctatg actagcaaag gagaagaact tttcactgga gttgtcccaa |
| 1021 | ttcttgttga attagatggt gatgttaatg ggcacaaatt ttctgtcagt ggagagggtg |
| 1081 | aaggtgatgc aacatacgga aaacttaccc ttaaatttat ttgcactact ggaaaactac |
| 1141 | ctgttccatg gccaacactt gtcactactt tctcttatgg tgttcaatgc ttttcaagat |
| 1201 | acccagatca tatgaaacgg catgactttt tcaagagtgc catgcccgaa ggttatgtac |
| 1261 | aggaaagaac tatatttttc aaggatgacg ggaactacaa gacacgtgct gaagtcaagt |
| 1321 | ttgaaggtga tacccttgtt aatagaatcg agttaaaagg tattgatttt aaagaagatg |
| 1381 | gaaacattct tggacacaaa ttggaataca actataactc acacaatgta tacatcatgg |
| 1441 | cagacaaaca aaagaatgga atcaaagtta acttcaaaat tagacacaac attgaagatg |
| 1501 | gaagcgttca actagcagac cattatcaac aaaatactcc aattggcgat ggccctgtcc |
| 1561 | ttttaccaga caaccattac ctgtccacac aatctgccct ttcgaaagat cccaacgaaa |
| 1621 | agagagacca catggtcctt cttgagtttg taacagctgc tgggattaca catggcatgg |
| 1681 | atgaactata caaataatac ccgggtaaat aatgcttatg tttttgtttg ctcctgttta |
| 1741 | gcaggtcgtt ccttcagcaa gaacaacaaa aatatgtgtt tttattgatc gttcaaacat |
| 1801 | ttggcaataa agtttcttaa gattgaatcc tgttgccggt cttgcgatga ttatcatata |
| 1861 | atttctgttg aattacgtta agcatgtaat aattaacatg taatgcatga cgttatttat |
| 1921 | gagatgggtt tttatgatta gagtcccgca attatacatt taatacgcga tagaaaacaa |
| 1981 | aatatagcgc gcaaactagg ataaattatc gcgcgcggtg tcatctatgt tactagatcg |
| 2041 | gcgcgccagc ttggcgtaat catggtcata gctgttgcga tcgctctgca gataacgcgt |
| 2101 | ggccggccat cttttatctt tagagttaag aactctttcg tattttggtg aggttttatc |
| 2161 | ctcttgagtt ttggtcatag acctattcat ggctctgata ccaattttta agcgggggct |
| 2221 | tatgcggatt atttcttaaa ttgataaggg gttattaggg ggtatagggt ataaatacaa |
| 2281 | gcattccctt agcgtatagt ataagtatag tagcgtacct ctatcaaatt tccatcttct |
| 2341 | taccttgcac agggcctgca accttatcct tccttgtctt cctccttcct tccgtccact |
| 2401 | tcatcatatt taaaccaaac ctacggggga gtcaacgtaa ccaaccctgc cttagcatct |
| 2461 | tttccctaac ggcctcctgc ctaagcggta cttctagctt cgaacggcgt ctgggctcca |
| 2521 | ggtttagtcg tctcgtgtct ggtttatatt cacgacaaag atctataggg actttaggag |
| 2581 | atctggattt tagtactgga ttttggtttt aggaattaga aattttattg atagaagtat |
| 2641 | tttacaaata caaatacata ctaagggttt cttatatgct caacacatga gcgaaaccct |
| 2701 | ataagaaccc taatttccct tatcgggaaa ctactcacac attatttatg gagaaaatag |
| 2761 | agagagatag atttgtagag agagactggt gatttcagcg aattcgagct ccccttactc |
| 2821 | gctttctttt tcgaaggtct cagtaccttc agggcatcct cttgatacat tactttccac |
| 2881 | ttcgattggg gcaagctgta gcagttcttg cttagaccga attgccatct cacagagatg |
| 2941 | ctgaagagtt cgcgaccctc cagaaacggt gatactaact cctcgaaacc gaatactata |
| 3001 | ggtacatccg atctggtcga aaccgaaaaa tcgagatgct gcatagttaa ccgaatctcc |
| 3061 | cgtccaagat ccaaggactc tgtgcagtga agcttccgtc ctgtcgtatc tgagatatct |
| 3121 | cttaaataca actttcccga aaccccagct ttccttgaaa ccaaggggat tatcttgatt |
| 3181 | cgaattcgtc tcatcgttat gtagccgcca ctcagtccaa ctcggacttt cgtcaggaag |
| 3241 | tttgaaggga gaagtggtac ctcctgatcc tccatcccaa cgttcactgt tagcttgttc |
| 3301 | cctagcgtcg tttccttgta tagctcgttc catatcgatt taaggggatc ctctagagtc |
| 3361 | gaagcttggg ctgtcctctc caaatgaaat gaacttcctt atatagagga agggtcttgc |
| 3421 | gaaggatagt gggattgtgc gtcatccctt acgtcagtgg agatgtcaca tcaatccact |
| 3481 | tgctttgaag acgtggttgg aacgtcttct ttttccacga tgctcctcgt gggtgggggt |
| 3541 | ccatctttgg gaccactgtc ggcagaggca tcttgaatga tagcctttcc tttatcgcaa |
| 3601 | tgatggcatt tgtaggagcc accttccttt tctactgtcc tttcgatgaa gtgacagata |
| 3661 | gctgggcaat ggaatccgag gaggtttccc gaaattaccc tttgttgaaa agtctcaata |
| 3721 | gccctttggt cttctgagac tgtatctttg acatttttgg agtagggggg taccgagctc |
| 3781 | gaattcggcc ggccctcact ggtgaaaaga aaaaccaccc cagtacatta aaaacgtccg |
| 3841 | caatgtgtta ttaagttgtc taagcgtcaa tttgtttaca ccacaatata tcctgccacc |
| 3901 | agccagccaa cagctccccg accggcagct cggcacaaaa tcaccactcg atacaggcag |
| 3961 | cccatcagtc cgggacggcg tcagcgggag agccgttgta aggcggcaga ctttgctcat |
| 4021 | gttaccgatg ctattcggaa gaacggcaac taagctgccg ggtttgaaac acggatgatc |
| 4081 | tcgcggaggg tagcatgttg attgtaacga tgacagagcg ttgctgcctg tgatcaaata |
| 4141 | tcatctccct cgcagagatc cgaattatca gccttcttat tcatttctcg cttaaccgtg |
| 4201 | acagagtaga caggctgtct cgcggccgag gggcgcagcc cctggggggg atgggaggcc |
| 4261 | cgcgttagcg ggccgggagg gttcgagaag ggggggcacc ccccttcggc gtgcgcggtc |
| 4321 | acgcgcacag ggcgcagccc tggttaaaaa caaggtttat aaatattggt ttaaaagcag |
| 4381 | gttaaaagac aggttagcgg tggccgaaaa acgggcggaa acccttgcaa atgctggatt |
| 4441 | ttctgcctgt ggacagcccc tcaaatgtca ataggtgcgc ccctcatctg tcagcactct |
| 4501 | gcccctcaag tgtcaaggat cgcgcccctc atctgtcagt agtcgcgccc ctcaagtgtc |
| 4561 | aataccgcag ggcacttatc cccaggcttg tccacatcat ctgtgggaaa ctcgcgtaaa |
| 4621 | atcaggcgtt ttcgccgatt tgcgaggctg gccagctcca cgtcgccggc cgaaatcgag |
| 4681 | cctgcccctc atctgtcaac gccgcgccgg gtgagtcggc ccctcaagtg tcaacgtccg |
| 4741 | cccctcatct gtcagtgagg gccaagtttt ccgcgaggta tccacaacgc cggcggccgc |
| 4801 | ggtgtctcgc acacggcttc gacggcgttt ctggcgcgtt tgcagggcca tagacggccg |
| 4861 | ccagcccagc ggcgagggca accagcccgg tgagcgtcgg aaaggcgctc ggtcttgcct |
| 4921 | tgctcgtcgg tgatgtacac tagtcgctgg ctgctgaacc cccagccgga actgacccca |
| 4981 | caaggcccta gcgtttgcaa tgcaccaggt catcattgac ccaggcgtgt tccaccaggc |
| 5041 | cgctgcctcg caactcttcg caggcttcgc cgacctgctc gcgccacttc ttcacgcggg |
| 5101 | tggaatccga tccgcacatg aggcggaagg tttccagctt gagcgggtac ggctcccggt |
| 5161 | gcgagctgaa atagtcgaac atccgtcggg ccgtcggcga cagcttgcgg tacttctccc |
| 5221 | atatgaattt cgtgtagtgg tcgccagcaa acagcacgac gatttcctcg tcgatcagga |
| 5281 | cctggcaacg ggacgttttc ttgccacggt ccaggacgcg gaagcggtgc agcagcgaca |
| 5341 | ccgattccag gtgcccaacg cggtcggacg tgaagcccat cgccgtcgcc tgtaggcgcg |
| 5401 | acaggcattc ctcggccttc gtgtaatacc ggccattgat cgaccagccc aggtcctggc |
| 5461 | aaagctcgta gaacgtgaag gtgatcggct cgccgatagg ggtgcgcttc gcgtactcca |
| 5521 | acacctgctg ccacaccagt tcgtcatcgt cggcccgcag ctcgacgccg gtgtaggtga |
| 5581 | tcttcacgtc cttgttgacg tggaaaatga ccttgttttg cagcgcctcg cgcgggattt |
| 5641 | tcttgttgcg cgtggtgaac agggcagagc gggccgtgtc gtttggcatc gctcgcatcg |
| 5701 | tgtccggcca cggcgcaata tcgaacaagg aaagctgcat ttccttgatc tgctgcttcg |
| 5761 | tgtgtttcag caacgcggcc tgcttggcct cgctgacctg ttttgccagg tcctcgccgg |
| 5821 | cggtttttcg cttcttggtc gtcatagttc ctcgcgtgtc gatggtcatc gacttcgcca |
| 5881 | aacctgccgc ctcctgttcg agacgacgcg aacgctccac ggcggccgat ggcgcgggca |
| 5941 | gggcaggggg agccagttgc acgctgtcgc gctcgatctt ggccgtagct tgctggacca |
| 6001 | tcgagccgac ggactggaag gtttcgcggg gcgcacgcat gacggtgcgg cttgcgatgg |
| 6061 | tttcggcatc ctcggcggaa aaccccgcgt cgatcagttc ttgcctgtat gccttccggt |
| 6121 | caaacgtccg attcattcac cctccttgcg ggattgcccc gactcacgcc ggggcaatgt |
| 6181 | gcccttattc ctgatttgac ccgcctggtg ccttggtgtc cagataatcc accttatcgg |
| 6241 | caatgaagtc ggtcccgtag accgtctggc cgtccttctc gtacttggta ttccgaatct |
| 6301 | tgccctgcac gaataccagc gaccccttgc ccaaatactt gccgtgggcc tcggcctgag |
| 6361 | agccaaaaca cttgatgcgg aagaagtcgg tgcgctcctg cttgtcgccg gcatcgttgc |
| 6421 | gccacatcta ggtactaaaa caattcatcc agtaaaatat aatattttat tttctcccaa |
| 6481 | tcaggcttga tccccagtaa gtcaaaaaat agctcgacat actgttcttc cccgatatcc |
| 6541 | tccctgatcg accggacgca gaaggcaatg tcataccact tgtccgccct gccgcttctc |
| 6601 | ccaagatcaa taaagccact tactttgcca tctttcacaa agatgttgct gtctcccagg |
| 6661 | tcgccgtggg aaaagacaag ttcctcttcg ggcttttccg tctttaaaaa atcatacagc |
| 6721 | tcgcgcggat ctttaaatgg agtgtcttct tcccagtttt cgcaatccac atcggccaga |
| 6781 | tcgttattca gtaagtaatc caattcggct aagcggctgt ctaagctatt cgtataggga |
| 6841 | caatccgata tgtcgatgga gtgaaagagc ctgatgcact ccgcatacag ctcgataatc |
| 6901 | ttttcagggc tttgttcatc ttcatactct tccgagcaaa ggacgccatc ggcctcactc |
| 6961 | atgagcagat tgctccagcc atcatgccgt tcaaagtgca ggacctttgg aacaggcagc |
| 7021 | tttccttcca gccatagcat catgtccttt tcccgttcca catcataggt ggtcccttta |
| 7081 | taccggctgt ccgtcatttt taaatatagg ttttcatttt ctcccaccag cttatatacc |
| 7141 | ttagcaggag acattccttc cgtatctttt acgcagcggt atttttcgat cagttttttc |
| 7201 | aattccggtg atattctcat tttagccatt tattatttcc ttcctctttt ctacagtatt |
| 7261 | taaagatacc ccaagaagct aattataaca agacgaactc caattcactg ttccttgcat |
| 7321 | tctaaaacct taaataccag aaaacagctt tttcaaagtt gttttcaaag ttggcgtata |
| 7381 | acatagtatc gacggagccg attttgaaac cacaattatg ggtgatgctg ccaacttact |
| 7441 | gatttagtgt atgatggtgt ttttgaggtg ctccagtggc ttctgtttct atcagctgtc |
| 7501 | cctcctgttc agctactgac ggggtggtgc gtaacggcaa aagcaccgcc ggacatcagc |
| 7561 | gctatctctg ctctcactgc cgtaaaacat ggcaactgca gttcacttac accgcttctc |
| 7621 | aacccggtac gcaccagaaa atcattgata tggccatgaa tggcgttgga tgccgggcaa |
| 7681 | cagcccgcat tatgggcgtt ggcctcaaca cgattttacg tcacttaaaa aactcaggcc |
| 7741 | gcagtcggta actatgcggt gtgaaatacc gcacagatgc gtaaggagaa aataccgcat |
| 7801 | caggcgctct tccgcttcct cgctcactga ctcgctgcgc tcggtcgttc ggctgcggcg |
| 7861 | agcggtatca gctcactcaa aggcggtaat acggttatcc acagaatcag gggataacgc |
| 7921 | aggaaagaac atgtgagcaa aaggccagca aaaggccagg aaccgtaaaa aggccgcgtt |
| 7981 | gctggcgttt ttccataggc tccgcccccc tgacgagcat cacaaaaatc gacgctcaag |
| 8041 | tcagaggtgg cgaaacccga caggactata aagataccag gcgtttcccc ctggaagctc |
| 8101 | cctcgtgcgc tctcctgttc cgaccctgcc gcttaccgga tacctgtccg cctttctccc |
| 8161 | ttcgggaagc gtggcgcttt ctcatagctc acgctgtagg tatctcagtt cggtgtaggt |
| 8221 | cgttcgctcc aagctgggct gtgtgcacga accccccgtt cagcccgacc gctgcgcctt |
| 8281 | atccggtaac tatcgtcttg agtccaaccc ggtaagacac gacttatcgc cactggcagc |
| 8341 | aggtaacctc gcgcatacag ccgggcagtg acgtcatcgt ctgcgcggaa atggacgggc |
| 8401 | ccccggcgcc agatctgggg aaccct |
| // | |

### REFERENCES

Fraenkel-Conrat, H. and Kimball, P. C. (1982) Virology. Prentice-Hall, New Jersey.

Lomonossoff, G. P. and Shanks, M. (1983). The nucleotide sequence of cowpea mosaic virus B RNA. EMBO Journal 2:2253-58.

Sainsbury, F. and Lomonossoff, G. P. (2008). Extremely high-level and rapid transient protein production in plants without the use of viral replication. Plant Physiology 148:1212-1218.

Sainsbury, F., Thuenemann, E. G. and Lomonossoff, G. P. (2009). pEAQ: versatile expression vectors for easy and quick transient expression of heterologous proteins in plants. Plant Biotechnology Journal 7:1-12.

Sainsbury, F., Canizares, M. C. and Lomonossoff, G. P. (2010). Cowpea mosaic virus: the plant virus-based biotechnology workhorse. Annual Review of Phytopathology 48:437-455.

Saxena, P., Hsieh, Y., Alvarado, V., Sainsbury, F., Saunders, K., Lomonossoff, G. P. and Scholthof, H. B. (2011). Improved foreign gene expression in plants using a virus-encoded suppressor of RNA silencing modified to be developmentally harmless. Plant Biotechnology Journal. In press.

Van Bokhoven H, Le Gall O, Kasteel D, Verver J, Wellink J, Van Kammen A. (1993). Cis- and trans-acting elements in cowpea mosaic virus RNA replication. Virology 195, 377-386.

1. A gene expression system comprising:
(a) a translational enhancer sequence,
which enhancer sequence consists or consists essentially of:
(i) the 5′ UTR of the RNA-1 genome segment of a bipartite RNA virus, or
(ii) a variant of said 5′ UTR, or
(iii) a portion of said 5′ UTR; and
(b) a heterologous gene encoding a protein of interest, wherein the gene is located downstream of the enhancer sequence,
wherein the enhancer and the gene encoding the protein of interest are operably linked to a plant promoter and terminator sequences and \or
wherein the heterologous gene does not encode any protein of the RNA-1 genome segment of a bipartite RNA virus.

2. A gene expression system as claimed in claim 1, wherein the enhancer and the gene encoding the protein of interest are operably linked to promoter and terminator sequences.

3. A gene expression system as claimed in claim 1, further comprising a 3′ UTR which is optionally derived from a 3′ UTR from the same bipartite RNA virus.

4. A gene expression system as claimed in claim 1, which includes an expression cassette having contiguously linked:
(a) a promoter, operably linked to
(b) a translational enhancer sequence,
which enhancer sequence consists or consists essentially of:
(i) the 5′ UTR of then RNA-1 genome segment of a bipartite RNA virus, or
(ii) a variant of said 5′ UTR, or
(iii) a portion of said 5′ UTR; and
(c) a heterologous gene encoding a protein of interest;
(d) a 3′ UTR;
(e) a terminator sequence.

5. A gene expression system comprising:
(a) a promoter, operably linked to
(b) a translational enhancer sequence,
which enhancer sequence consists or consists essentially of:
(i) the 5′ UTR of then RNA-1 genome segment of a bipartite RNA virus, or
(ii) a variant of said 5′ UTR, or
(iii) a portion of said 5′ UTR; and
(c) a heterologous sequence for facilitating insertion of a gene encoding a protein of interest into the gene expression system, wherein the heterologous sequence is located downstream of the enhancer sequence; and optionally
(d) a 3′ UTR;
(e) a terminator sequence
wherein the promoter is a plant active promoter and\or
wherein the heterologous sequence gene does not encode any protein of the
RNA-1 genome segment of a bipartite RNA virus.

6. A gene expression system as claimed in claim 5, which comprises the sequence shown in Sequence Annex II

7. A gene expression system as claimed in claim 1, which is present in an expression vector.

8. A gene expression system as claimed in claim 2, wherein the enhancer and the gene encoding the protein of interest is operably linked to a plant promoter and terminator sequences and wherein the heterologous gene or sequence does not encode any protein of the RNA-1 genome segment of a bipartite RNA virus.

9. A gene expression system as claimed in 7, wherein the vector further includes a suppressor of gene silencing operably linked to promoter and terminator sequences.

10. A gene expression system as claimed in claim 9, wherein the suppressor of gene silencing is the p19 protein or a variant thereof.

11. A gene expression system as claimed in claim 7, wherein the vector is a plant binary vector.

12. A gene expression system as claimed in claim 11, wherein the vector is shown in FIG. 1a.

13. A gene expression system as claimed in claim 1, wherein the bipartite RNA virus is a member of the Comovirinae, more preferably a comovirus.

14. A gene expression system as claimed in claim 13, wherein the bipartite RNA virus is cowpea mosaic virus.

15. A gene expression system as claimed in claim 14, wherein the enhancer sequence consists or consists essentially of:
(i) the 206 nucleotide 5′ UTR of the CPMV RNA-1 genome segment shown in Sequence Annex I, or
(ii) a variant of said 5′ UTR having at least 99%, 98%, 97%, 96%, 95%, 90%, 85%, 80%, 75%, 70%, 65%, 60%, 55%, or 50% identity to said 5′ UTR;
(iii) a portion of said 5′ UTR having at least 50, 60, 70, 80, 90, 100, 110, 120, 130, 140, 150, 160, 170, 180, 190, 200, 205 contiguous nucleotides thereof.

16. A gene expression system as claimed in claim 14, comprising a 3′ UTR downstream of the heterologous gene or heterologous sequence and upstream of a terminator sequence, wherein the 3′UTR sequence consists or consists essentially of:
(i) the 84 nucleotide 3′ UTR of the CPMV RNA-1 genome segment shown in Sequence Annex I, or
(ii) a variant of said 3′ UTR having at least 99%, 98%, 97%, 96%, 95%, 90%, 85%, 80%, 75%, 70%, 65%, 60%, 55%, or 50% identity to said 3′ UTR;
(iii) a portion of said 3′ UTR having at least 50, 60, 70, 80 contiguous nucleotides thereof.

17. A method for expressing a protein of interest in a host cell or organism using a gene expression system as claimed in claim 1.

18. A method as claimed in claim 17, wherein the host organism is a eukaryotic host, which is optionally a plant.

19. A method as claimed in claim 18, comprising the step of introducing the gene expression system into the host organism.

20. A method as claimed in claim 19, wherein the gene encoding the protein of interest is transiently expressed in the host.

21. A method as claimed in 17, wherein the protein of interest is a first protein, and the system is introduced into the host with further gene expression system encoding a second protein, which further gene expression system comprises:
(i) a promoter, operably linked to
(ii) an expression enhancer sequence derived from the RNA-2 genome segment of a bipartite RNA virus, in which a target initiation site in the RNA-2 genome segment has been mutated.
(iii) a gene encoding the second protein;
(iv) a terminator sequence; and optionally
(v) a 3′ UTR located upstream of said terminator sequence.

22. A method as claimed in claim 21, wherein it is desired to express the first and second proteins at differing levels of expression and\or wherein the first and second proteins are part of a single pathway or complex.

23. A method as claimed in claim 17, wherein a suppressor of gene silencing is also introduced into said host, which suppressor is optionally the p19 protein or a variant thereof.

24. A host obtained or obtainable by a method as claimed in claim 20.

25. A host transiently or stably transfected with a gene expression system as claimed in claim 1.

26. A host as claimed in claim 25, wherein the host is a plant or plant cell.

27. A transgenic host as claimed in claim 25, which also includes in its genome a further gene expression system encoding a second protein, which further gene expression system comprises:
(i) a promoter, operably linked to
(ii) an expression enhancer sequence derived from the RNA-2 genome segment of a bipartite RNA virus, in which a target initiation site in the RNA-2 genome segment has been mutated.
(iii) a gene encoding the second protein;
(iv) a terminator sequence; and optionally
(v) a 3′ UTR located upstream of said terminator sequence.

28. A transgenic host as claimed in claim 27, which also includes in its genome a heterologous suppressor of gene silencing, which is optionally the p19 protein or a variant thereof.

29. A method for generating one or more proteins of interest, comprising using a host as claimed in claim 25 and harvesting a tissue in which the protein of interest has been expressed and isolating the protein of interest from the tissue.


SEQUENCE LISTING (3 sequences)

SEQ ID NO: 1 - DNA, 5889 bp, Cowpea mosaic virus
tattaaaatcaatacaggttttgataaaagcgaacgtggagaaatccaaacctttctttctttcctcaatctcttcaattgcgaacgaaatccaagctttggttttgctgaaacaaatacacaacgtatactgaatttggcaaatttctctctctctctctgtcattttctttcttctgtcgggactttcttagtcttgacccaacatgggtctcccagaatatgaggccgatagtgaggctttattaagtcaactcactatcgaattcacacccggcatgacagtttcttcattgttggcacaagtcaccactaatgactttcacagtgccattgagttttttgctgcagaaaaagcagtagacattgagggcgttcattacaatgcgtatatgcaacaaattaggaaaaaccctagtttattacgcatttccgtggtagcttatgctttccacgtttcagacatggtagctgagaccatgtcttatgatgtttatgaatttctgtataaacattatgcccttttcatctctaatctggtgaccagaacactcagatttaaagagcttttgctgttctgtaagcagcaatttctggagaaaatgcaagcttcaatagtctgggctccggaacttgagcaatatcttcaagttgaaggggatgctgtggctcaaggagtttcacaactgttatacaagatggtcacttgggtgcccacttttgtcagaggagcagtagactggagcgttgatgcgattttggtcagtttcaggaaacattttgaaaagatggttcaggagtatgtgcccatggctcatcgcgtttgcagttggctgagccaactatgggataagatcgtgcaatggatctcacaagcaagtgagaccatgggttggtttctagatggttgtcgggatttgatgacttggggaattgccactctcgcaacatgtagtgctctctccctggttgagaagctgttagtcgcaatgggttttctggttgagcctttcggcttgagtggaatcttcttgcggacgggagttgttgcggcagcttgttataactatgggactaattctaagggttttgccgagatgatggctttgttgtcattggcggctaactgtgtctctacagttatagttggtggctttttccctggtgaaaaggacaatgcacagagtagtcctgttatcctcttagaaggattggctgggcagatgcaaaacttttgtgagactacacttgtcagtgttgggaaaacatgcactgccgtcaatgctatctcaacatgttgtgggaatctgaaagcactggccggaaggatcttgggcatgctcagagattttatctggaagactttgggctttgagaccagatttctagcagatgcatctttgctttttggcgaggatgttgatggatggctcaaagcaatcagtgatctgcgagatcaatttattgccaaatcatactgttcgcaggatgagatgatgcagattttggtgttgcttgaaaagggaaggcagatgcggaaaagtggtctttctaaaggaggcatttctcctgctatcattaatctgattctcaaagggattaatgatcttgaacaattgaaccgcagctgttcagtgcaaggagtaagaggagttaggaaaatgccatttaccattttcttccaaggaaagtcacgcactggtaagagtttgctgatgagtcaggttacaaaggattttcaggatcactatggattgggtggagaaactgtgtacagtagaaatccttgtgatcaatattggagtggatatcggcggcaaccttttgtgctgatggatgattttgccgccgttgttactgagccgtctgctgaggctcagatgatcaatctgatttctagtgctccatatcctttgaatatggctggacttgaagaaaaaggaatttgttttgattctcaatttgtttttgtttccaccaacttcttggaagtatctcctgaagccaaagttagggacgatgaggctttcaagaacaggagacatgtgattgttcaggtttcaaatgatcctgccaaagcatatgatgctgcaaattttgctagcaaccaaatttacaccattttggcatggaaggatggtcgatacaacaccgtgtgcgttattgaggactatgatgagctggtggcatatttgttgactaggagtcaacagcatgctgaagagcaggagaagaatcttgctaacatgatgaagagtgctacatttgaaagtcatttcaaaagtttagttgaagtccttgagctcggttctatgatatctgctggttttgatatcattcggccagaaaaacttcctagtgaagctaaggagaagagagtcctttacagtattccctacaatggggagtattgtaatgcactcattgatgacaattacaatgttacttgctggtttggtgagtgtgttggtaatcctgagcagctctctaagtacagtgaaaagatgcttttgggtgcttatgaatttcttctgtgttctgagagcttgaatgttgtaattcaggcacatttgaaggaaatggtttgccctcaccattatgacaaggagctcaattttattggcaagataggagagacctactatcacaatcagatggtttcaaatatcggctctatgcagaaatggcatcgtgccattctgtttggaattggggttctcttgggaaaggaaaaagagaagacatggtaccaagttcaggttgccaatgttaaacaagctctttacgacatgtacactaaggagattcgtgattggcccatgccgatcaaagtcacctgtggaattgtcttggcagctattgggggtagtgccttttggaaagtgtttcaacaactagtgggaagcggaaatggtccagtattgatgggtgtggctgctggagcattcagtgctgagcctcaaagtagaaagcccaataggtttgatatgcagcaatacaggtacaacaatgttcctctcaagagaagagtttgggcagacgcacaaatgtctttggatcagagtagtgttgctatcatgtctaagtgtagggctaatctggtttttggaggcactaatttgcaaatagtcatggtaccaggaagacgctttttggcatgcaaacatttcttcacccacataaagaccaaattgcgtgtggaaatagttatggatggaagaaggtactatcatcaatttgatcctgcaaatatttatgatatacctgattctgagttggtcttgtactcccatcctagcttggaagacgtttcccattcttgctgggatctgttctgttgggacccagacaaagaattgccttcagtatttggagcggatttcttgagttgtaaatacaacaagtttgggggtttttatgaggcgcaatatgctgatatcaaagtgcgcacaaagaaagaatgccttaccatacagagtggtaattatgtgaacaaggtgtctcgctatcttgagtatgaagctcctactatccctgaggattgtggatctcttgtgatagcacacattggtgggaagcacaagattgtgggtgttcatgttgctggtattcaaggtaagataggatgtgcttccttattgccaccattggagccaatagcacaagcgcaaggtgctgaggaatactttgattttcttccagctgaagagaatgtatcttctggagtggctatggtagcaggactcaaacaaggagtttacataccattacccacaaaaacagcgctagtggagaccccctccgagtggcatttggacacaccatgtgacaaagttcctagcattttagttcccacggatccccgaattcctgcgcaacatgaaggatatgatcctgctaagagtggggtttccaagtattcccagcctatgtctgctctggaccctgagttacttggcgaggtggctaatgatgttctcgagctatggcatgactgcgctgtagattgggacgattttggtgaagtgtctctggaggaagctttgaatggatgtgaaggagtggaatatatggaaaggattccattagcaacttctgagggctttccgcacattctttctagaaatgggaaagaaaaggggaaaagacggtttgttcagggagatgattgtgttgtctcactaattccaggaactactgtagccaaagcttatgaggagttggaagcaagtgcacacagatttgttcccgctcttgttgggattgaatgtccaaaagatgagaagttgcctatgagaaaggtttttgataagcctaagaccaggtgttttaccattttgccaatggaatataatttggtcgttcgtaggaagtttctgaattttgtgcgctttatcatggccaatcgtcacagactcagttgtcaagtgggtattaatccatattcaatggaatggagtcgcttagcagcaaggatgaaagagaaaggcaatgatgtcttgtgttgtgattatagctcattcgatggcttgctttctaagcaagtgatggatgtcattgctagcatgatcaatgaactttgtggtggagaggatcaactcaaaaatgcaaggcgaaacttgttaatggcgtgttgctctaggttggctatttgcaagaatacagtatggagagttgagtgtggtattccttcagggtttccaatgacagtgattgtgaatagcatttttaatgagattctcattcgctatcattacaagaaactcatgcgcgaacaacaagctcctgaactgatggtacagagttttgataaactcatagggctggtgacttatggtgatgataatctgatttcagtgaatgctgttgtgacaccctattttgatgggaagaaattgaagcaatctttggctcagggtggtgtgactatcactgatggtaaggacaaaacaagtttggaacttccttttcgcagattggaagaatgtgattttctcaagagaacttttgttcagaggagcagtaccatctgggacgctccagaggataaggcaagtttgtggtcgcagcttcattatgttaattgcaacaattgtgagaaagaagttgcttatttgactaatgttgttaatgttcttcgtgaactttatatgcatagtcctcgggaagccacagaatttaggaggaaggtcttaaagaaggtcagttggatcactagtggagatttgcctactttggcacaattgcaagagttctatgagtaccagcggcagcaaggtggggcagacaacaatgacacttgtgacttgttaacaagtgtagacttgctaggtcctcctttgtcttttgagaaagaagcgatgcacggatgcaaagtgtctgaagaaatcgtcaccaagaatttggcatattacgatttcaaaaggaaaggtgaggatgaagtggtatttctgttcaatacgctctatcctcagagttcattgcctgatgggtgtcactctgtgacctggtctcagggtagtggaaggggaggtttgcccacacaaagttggatgagctataatataagcaggaaagattctaatatcaacaagattattagaactgctgtttcttcgaagaaacgagtgatattctgtgctcgtgataatatggttcctgttaacattgtagctttgctctgtgctgttagaaacaagctgatgcccactgctgtatctaatgctacacttgtcaaggtgatggaaaatgccaaagctttcaagtttttaccagaagagttcaatttcgctttttctgatgtttaggtaaataatgcttatgtttttgtttgctcctgtttagcaggtcgttccttcagcaagaacaacaaaaatatgtgtttttatt

SEQ ID NO: 2 - DNA, 908 bp, Artificial sequence
  Synthetic sequence RT expression cassette.
ggaaacctcctcggattccattgcccagctatctgtcactttattgagaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcaatacaggttttgataaaagcgaacgtggagaaatccaaacctttctttctttcctcaatctcttcaattgcgaacgaaatccaagctttggttttgctgaaacaaatacacaacgtatactgaatttggcaaatttctctctctctctctgtcattttctttcttctgtcgggactttcttagtcttgacccaaccctcgagcctgcaggcaattgtatacctaggtccggaccggtacgtacccgggtaaataatgcttatgtttttgtttgctcctgtttagcaggtcgttccttcagcaagaacaacaaaaatatgtgtttttattgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 3 - DNA, 8426 bp, Artificial sequence
  Synthetic sequence pEAQexpress-RT-GFP
gtggttggcatgcacatacaaatggacgaacggataaaccttttcacgcccttttaaatatccgattattctaataaacgctcttttctcttaggtttacccgccaatatatcctgtcaaacactgatagtttgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgttaattaaggaaacctcctcggattccattgcccagctatctgtcactttattgagaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcaatacaggttttgataaaagcgaacgtggagaaatccaaacctttctttctttcctcaatctcttcaattgcgaacgaaatccaagctttggttttgctgaaacaaatacacaacgtatactgaatttggcaaatttctctctctctctctgtcattttctttcttctgtcgggactttcttagtcttgacccaaccctcgagctatgactagcaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaaacggcatgactttttcaagagtgccatgcccgaaggttatgtacaggaaagaactatatttttcaaggatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataatacccgggtaaataatgcttatgtttttgtttgctcctgtttagcaggtcgttccttcagcaagaacaacaaaaatatgtgtttttattgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccagcttggcgtaatcatggtcatagctgttgcgatcgctctgcagataacgcgtggccggccatcttttatctttagagttaagaactctttcgtattttggtgaggttttatcctcttgagttttggtcatagacctattcatggctctgataccaatttttaagcgggggcttatgcggattatttcttaaattgataaggggttattagggggtatagggtataaatacaagcattcccttagcgtatagtataagtatagtagcgtacctctatcaaatttccatcttcttaccttgcacagggcctgcaaccttatccttccttgtcttcctccttccttccgtccacttcatcatatttaaaccaaacctacgggggagtcaacgtaaccaaccctgccttagcatcttttccctaacggcctcctgcctaagcggtacttctagcttcgaacggcgtctgggctccaggtttagtcgtctcgtgtctggtttatattcacgacaaagatctatagggactttaggagatctggattttagtactggattttggttttaggaattagaaattttattgatagaagtattttacaaatacaaatacatactaagggtttcttatatgctcaacacatgagcgaaaccctataagaaccctaatttcccttatcgggaaactactcacacattatttatggagaaaatagagagagatagatttgtagagagagactggtgatttcagcgaattcgagctccccttactcgctttctttttcgaaggtctcagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagtggtacctcctgatcctccatcccaacgttcactgttagcttgttccctagcgtcgtttccttgtatagctcgttccatatcgatttaaggggatcctctagagtcgaagcttgggctgtcctctccaaatgaaatgaacttccttatatagaggaagggtcttgcgaaggatagtgggattgtgcgtcatcccttacgtcagtggagatgtcacatcaatccacttgctttgaagacgtggttggaacgtcttctttttccacgatgctcctcgtgggtgggggtccatctttgggaccactgtcggcagaggcatcttgaatgatagcctttcctttatcgcaatgatggcatttgtaggagccaccttccttttctactgtcctttcgatgaagtgacagatagctgggcaatggaatccgaggaggtttcccgaaattaccctttgttgaaaagtctcaatagccctttggtcttctgagactgtatctttgacatttttggagtaggggggtaccgagctcgaattcggccggccctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacagagtagacaggctgtctcgcggccgaggggcgcagcccctgggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcggtcttgccttgctcgtcggtgatgtacactagtcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaactatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcaggtaacctcgcgcatacagccgggcagtgacgtcatcgtctgcgcggaaatggacgggcccccggcgccagatctggggaaccct